(19) United States
(12) Patent Application Publication
(10) Pub. No.: US 2023/0016828 A1
(43) Pub. Date: Jan. 19, 2023

(54) METHOD AND SYSTEM FOR MANAGING DATA EXCHANGE IN THE CONTEXT OF A MEDICAL EXAMINATION

(71) Applicant: E-SCOPICS, Aix-en-Provence (FR)

(72) Inventors: Claude COHEN-BACRIE, Aix-en-Provence (FR); Adrien BESSON, Marseille (FR); Frederic WINTZENRIETH, Aix-en-Provence (FR); Luc BERTRAND, Marseille (FR); Eric GUIFFARD, La Roque D'Antheron (FR)

(21) Appl. No.: 17/786,195

(22) PCT Filed: Dec. 21, 2020
(86) PCT No.: PCT/EP2020/087458

§ 371 Date: Jun. 16, 2022

(30) Foreign Application Priority Data
Dec. 20, 2019 (FR) FR1915204

(51) Int. Cl. H04L 9/32; H04L 9/08; H04L 9/40; G16H 40/60

(52) U.S. Cl. CPC H04L 9/3263; H04L 9/0825; H04L 63/0869; G16H 40/60

(57) ABSTRACT

The invention relates to a method for managing exchanges of data between: —a probe (1) comprising a memory containing a probe digital certificate including a probe public key, —a terminal (2) comprising a memory containing a terminal digital certificate including a terminal public key, —a remote platform (3) configured to: ^{∘}deliver the probe digital certificate to the probe and ^{∘}deliver the terminal digital certificate to the terminal, characterised in that the method comprises the implementation of an authentication procedure consisting of the following steps:—a first step in which the probe verifies the identity of the terminal from the terminal digital certificate; —a second step in which the terminal verifies the identity of the probe from the probe digital certificate, and—a third step in which the probe, the terminal and the platform each generate an identical session key from the probe and terminal public keys.

This invention relates to the general technical field of services security.

In particular, this invention relates to a method allowing:
the authentication of entities exchanging data—for example data of a medical nature and/or control data and/or monitoring data—in a communication network, and
the encryption of the exchanged data in order to guarantee their confidentiality on the one hand and their reliability on the other.

The invention also relates to an associated system of authentication and encryption.

More precisely, the invention relates to an advanced application of data acquisition and transfer solutions: dematerialized ultrasonography.

General Overview of the Prior Art

Ultrasound imagery is used in many diagnostic procedures due to its non-invasive nature, its relatively low cost and the absence of exposure of the patient to harmful ionizing radiation.

In the past few years, new so-called “ultra-portable” ultrasonography solutions have been developed in which data acquired by a probe—and optionally processed—are transmitted on a remote storage and/or processing platform—known under the name of “cloud”—using a communication network—such as the Internet network.

Indeed, the cloud makes it possible to pool data retention and processing structures, and has very significant computational power.

However, the transmission of data—acquired medical data, processed medical data, system data representative of control/monitoring data of the probe etc. —via a computer network (such as the Internet) raises the crucial problem of the integrity, authenticity and inviolability of these data, in order to ensure:
the integrity of the system data: for example the instructions for driving the probe must not be able to be modified by a malicious third-party in order to eliminate the risks to patient health,
the confidentiality of medical data which must not be accessible by a malicious third party: only the authorized medical personal (doctor etc.) must have access to these medical data, and
the reliability of medical data: all the components (i.e. acquired data, processed data, measurements, etc.) of the examination must be consistent (for example medical data from a previous examination must not interfere with the medical data of an examination in progress/all acquired medical data of an examination in progress must be processed/etc.).

An aim of this invention is to provide a method and a system for performing an ultrasonic examination in a situation of mobility and incorporating a solution to the aforementioned problems of integrity, reliability and confidentiality related to the exchange of data via a computer network such as the Internet.

Overview of the Invention

For this purpose, the invention relates to a method of management of the data exchanges during a procedure of medical examination of a patient, the method allowing the management of the data exchanges between:
a data acquisition probe, said probe including a memory containing a probe digital certificate including a probe public key,
a terminal able to communicate with the probe via wired or wireless communication means, said terminal including a memory containing a terminal digital certificate including a terminal public key,
a remote platform able to communicate with the terminal via a computer network such as the Internet, said platform being configured to:
issue the probe digital certificate to the probe,
issue the terminal digital certificate to the terminal, noteworthy in that the method comprises, prior to the implementation of the examination procedure, the implementation of an authentication procedure comprising the following phases:
a first phase wherein the probe verifies the identity of the terminal on the basis of the terminal digital certificate, and
a second phase wherein the terminal verifies the identity of the probe on the basis of the probe digital certificate
a third phase wherein the probe, the terminal and the platform each generate an identical session key (independently), said session key being produced on the basis of the probe and terminal public keys (and not being transmitted between the probe, the terminal and the platform).

This session key is used to symmetrically encrypt session data transmitted between the probe, the terminal and the platform once the authentication procedure is complete.

In the context of this invention, the exchanged session data may consist in:
system data including:
   control data (i.e. driving instructions) of the probe and/or the terminal and/or the platform, and/or
   monitoring data of the probe and/or the terminal and/or the platform;
medical data including:
   medical data acquired (and/or pre-processed) by the probe, and/or
   medical data processed by the terminal and/or the remote platform.

These session data can be encrypted/decrypted by the probe or the terminal or the remote platform using the session key. The information contained in these session data is therefore accessible by all three entities of the system.

Preferred, but non-limiting embodiments of the invention are as follows:
the identical session key is generated independently by the probe, the terminal and the platform, the probe and terminal public keys being stored respectively:
in the memory of the probe,
in the memory of the terminal, and
in a storage unit of the platform

such that said session key is not exchanged between the probe, the terminal and the platform via the communication means and/or the computer network;
the session key is used to encrypt according to a symmetrical cryptography mode data exchanged between the probe, the terminal and the platform during the implementation of the examination;
the first phase comprises the following steps:
transmission by the terminal of a pairing request, the pairing request containing the terminal digital certificate,
reception by the probe of the pairing request and extraction of the terminal digital certificate,
verification by the probe of the terminal digital certificate by certifying the authenticity of said terminal digital certificate using a platform public key contained in the memory of the probe;
the first phase further comprises the following steps:
if the terminal digital certificate is authentic, then exchange of verification information between the probe and the terminal, said verification information being successively encrypted and decrypted using the terminal public key and a terminal private key stored in the memory of the terminal,
if the terminal digital certificate is not authentic, then transmission, by the probe, of an alert message.
the step of exchanging verification information comprises the sub-steps consisting in:
   extraction, by the probe, of the terminal public key contained in the terminal digital certificate,
   generation, by the probe, of a verification information,
   asymmetrical encryption, by the probe, of the verification information with the terminal public key,
   sending, by the probe, of an answer message including the verification information encrypted with the terminal public key,
   reception, by the terminal, of the answer message and decryption of the verification information using a terminal private key stored in the memory of the terminal,
   sending, by the terminal, of a confirmation message containing the decrypted verification information,
   reception, by the probe, of the confirmation message,
   comparison, by the probe, of the decrypted verification information contained in the confirmation message with the verification information contained in the answer message transmitted by the probe, to define whether said verification information are identical or different, and
   if the verification information are identical, transmission of a validation message representative of a successful authentication,
   if the verification information are different, transmission of an alert message representative of a failed authentication.
the second phase comprises the following steps:
reception, by the terminal, of a result message transmitted by the probe, the probe digital certificate being incorporated into the result message,
extraction, by the terminal, of the probe digital certificate,
verification by the terminal of the authenticity of the probe digital certificate by comparing the signature of said terminal digital certificate with a platform public key contained in the memory of the terminal;
the second phase further comprises the following steps:
if the probe digital certificate is authentic, then exchange of verification information between the probe and the terminal, said verification information being successively encrypted and decrypted using the probe public key and a probe private key stored in a memory of the probe,
if the probe digital certificate is not authentic, then transmission, by the terminal, of an alert message;
the step of exchange of verification information comprises the sub-steps consisting in:
   extraction, by the terminal, of the probe public key contained in the probe digital certificate,
   generation, by the terminal, of a verification information,
   asymmetrical encryption, by the terminal, of the verification information using the probe public key,
   sending, by the terminal, of a justification message including the verification information encrypted with the probe public key,
   reception, by the probe, of the justification message and decryption of the verification information using a probe private key stored in the memory of the probe,
   sending, by the probe, of a proof message containing the decrypted verification information,
   reception, by the terminal, of the proof message,
   comparison, by the terminal, of the verification information contained in the proof message with the verification information contained in the justification message transmitted by the terminal, to define whether said verification information are identical or different, and
   if the verification information are identical, transmission of a validation message representative of a successful authentication,
   if the verification information are different, transmission of an alert message representative of a failed authentication;
the method comprises, prior to the implementation of an authentication procedure, a subscription procedure wherein:
the terminal sends the platform an examination request message including a probe identifier, a terminal identifier, and the terminal public key,
the platforms sends the terminal a pairing authorization message including a platform certificate including a platform public key and a terminal certificate including the terminal public key.

The invention also relates to a system for the exchange of data during a procedure of examination of a patient, the system comprising:
a data acquisition probe, said probe including a memory containing a probe digital certificate including a probe public key,
a terminal able to communicate with the probe via wired or wireless communication means, said terminal including a memory containing a terminal digital certificate including a terminal public key,
a remote platform able to communicate with the terminal via a computer network such as the Internet, said platform being configured to:
   issue the probe digital certificate to the probe,
   issue the terminal digital certificate to the terminal,

noteworthy in that the probe, the terminal and platform comprise means suitable for the implementation of an authentication procedure, prior to the implementation of the examination procedure, the authentication procedure comprising the following phases:
a first phase wherein the probe verifies the identity of the terminal on the basis of the terminal digital certificate, and
a second phase wherein the terminal verifies the identity of the probe on the basis of the probe digital certificate
a third phase wherein the probe, the terminal and the platform each generate an identical session key, said session key being produced on the basis of the probe and terminal public keys.

Advantageously, the probe, the terminal and the platform comprise means for the implementation of the phases, steps and sub-steps of the management method defined above.

### OVERVIEW OF THE FIGURES

Other features, aims and advantages of this invention will become apparent from the following description, which is purely illustrative and non-limiting and must be read with reference to the appended drawings wherein:

FIG. 1 is a schematic representation of a system for the exchange of data (i.e. control data, monitoring data and/or data of a medical nature) during a procedure of examination of a patient,

FIG. 2 is a schematic representation of a first phase of dialog between a probe and a terminal, said first phase being implemented during an authentication procedure,

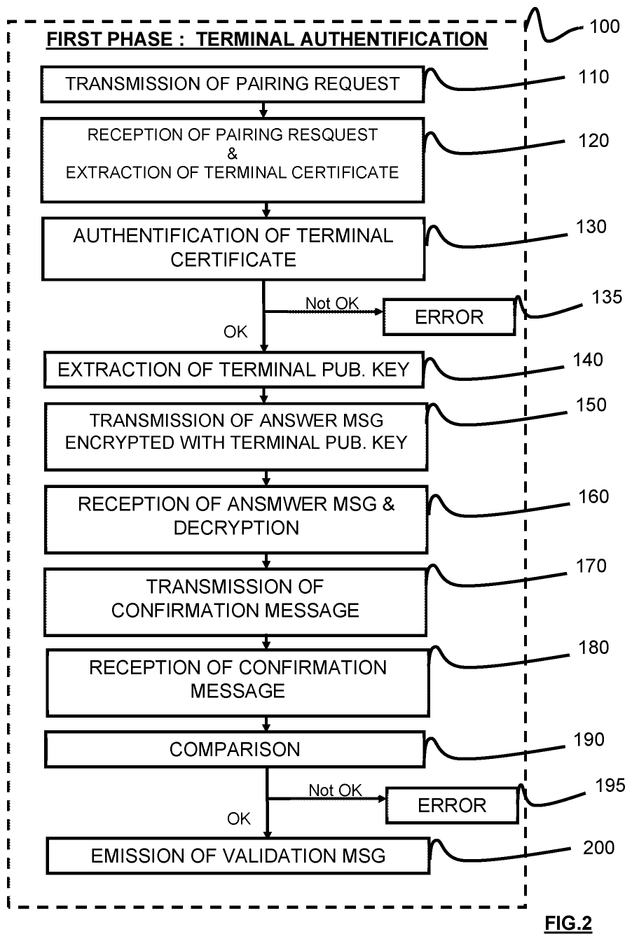

FIG. 3 is a schematic representation of a second phase of dialog between the probe and the terminal, the second phase being implemented during the authentication procedure.

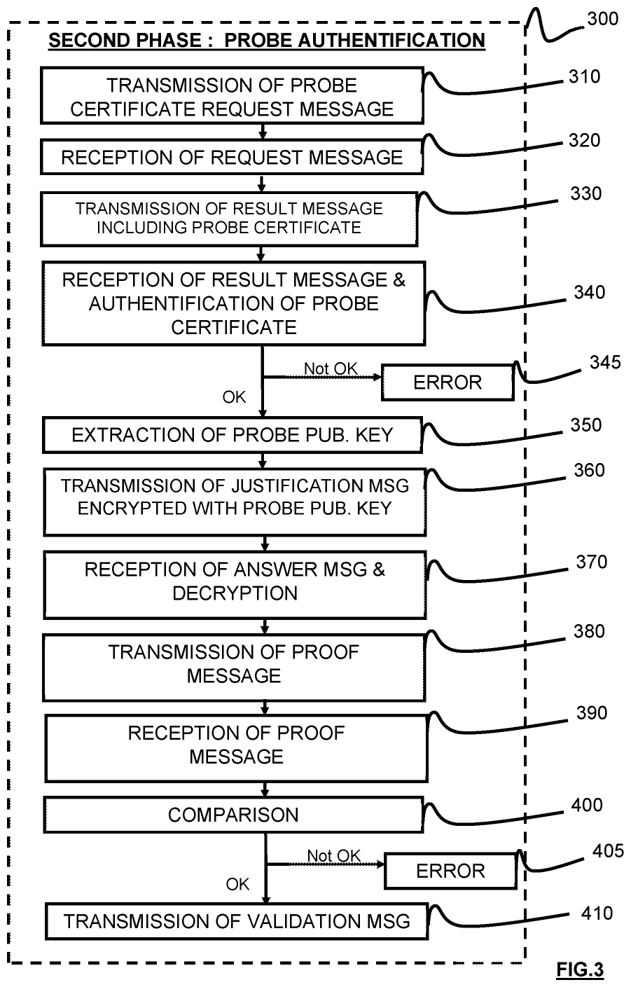

### DESCRIPTION OF THE INVENTION

1. General

1.1. Encryption

The invention described in the remainder of the text uses the data cryptography technique which allows a transmitting entity to encrypt the transmitted data such that only an authentic receiving entity can decrypt these data in order to interpret them.

1.1.1. Principle of Symmetrical/Asymmetrical Encryption

In a known manner, a distinction is made between symmetrical cryptography, in which a same secret key serves to encrypt and decrypt the exchanged data, and asymmetrical cryptography, in which a pair of separate keys—the so-called “public key” and “private key”—are used.

Symmetrical cryptography is suitable for a dialog within a single emitter/receiver pair with reciprocal trust since the emitter and the receiver secretly share the same key.

Asymmetrical cryptography is more suitable for setting up a dialog with many potential participants.

As a reminder, it is recalled for information purposes that when the private key is held by the receiving entity, any transmitting system can encrypt a datum by means of the public key and transmit it to the receiving entity: only the receiving entity can decrypt the datum by means of the private key. This ensures the confidentiality of the transmitted document.

Conversely, when the private key is held by the transmitting entity, it is the only one to be able to encrypt the datum. Any receiving entity can decrypt the datum using the public key. It can do this with the assurance that the transmitting entity that has transmitted the datum is the entity that has the private key.

1.1.2. Principle of Encryption by Certificates

One drawback of the use of the asymmetrical encryption technique comes from the transmission of the public key. If it is not secure, a malicious third-party entity can be positioned between a trusted entity and its public by distributing false public keys (via a fake website for example) then intercept all communications, allowing it to usurp the identity of the trusted entity. This type of attack is in particular known by the name of “man-in-the-middle attack”.

This is why in the context of the present invention, the method and the system described in the remainder of the text are based on the use of electronic certificates which are better suited to the needs of the concerned application. Specifically, as will become apparent further on, the invention implements data exchanges:
between at least three distinct entities,
the authenticity of one of these entities must be validated by the two other entities prior to the exchange of any datum.

As a reminder, an electronic certificate (also known as a “digital certificate” or a “public key certificate”) constitutes a “digital identity card” used to:
identify and authenticate an entity, but also to
encrypt data exchanges.

An electronic certificate is composed of a set of data containing:
one or more public keys,
information identifying the entity associated with the certificate (for example: name, location, and electronic address of the entity that transmitted the certificate);
at least one signature constructed on the basis of a private key of the entity that generated the certificate; thus, the signing entity is the only authority making it possible to trust (or not) the accuracy of the information of the certificate.

Such an electronic certificate is therefore:
unfalsifiable: it is encrypted to prevent any modification.
nominative: it is issued to an entity and constitutes the digital identity card of this entity,
certified; it is signed by the entity that issued it.

1.2. Hardware Architecture

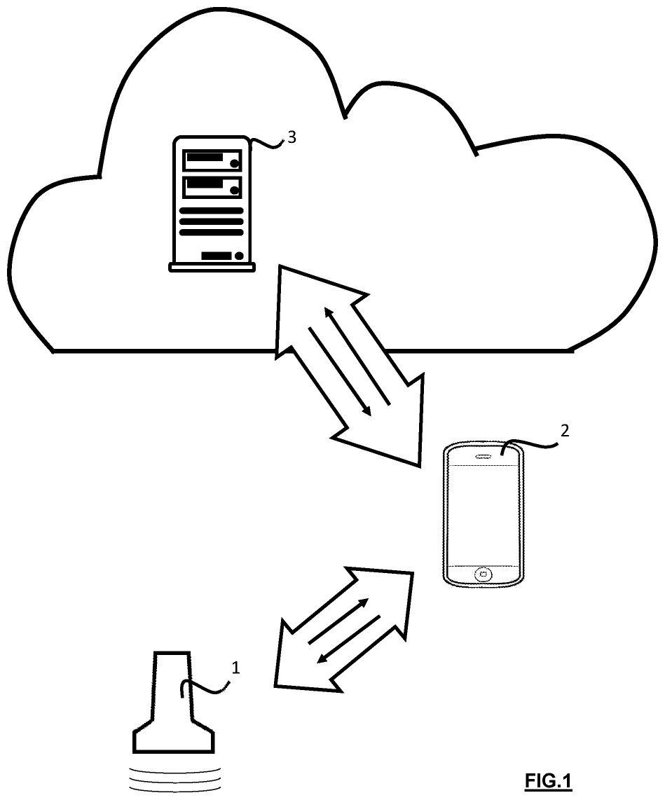

With reference to FIG. 1, an example of a system has been illustrated in which the authentication method described in the remainder of the text can be implemented prior to the exchange of data between the different entities of the system.

The system comprises three separate entities:
an acquisition probe 1,
a local terminal 2 connected to the probe by wired or wireless communication means, and
a remote platform 3 connected to the terminal 2 via a computer network such as the Internet.

1.2.1. Probe

The probe 1 allows the registration of medical data representative of a region of interest of a patient (internal structures, organ, etc.).

The probe 1 is for example an ultrasound probe including:
a plurality of ultrasonic transducers for the transmission of ultrasonic waves, the reception of ultrasonic echoes and the conversion of the received ultrasonic echoes into electrical signals forming the data,
a calculator for the optional pre-processing of data,
a communication unit (wired or wireless) for the transmission of the acquired data to the external terminal.

The processing of the data acquired by the probe 1 allows the extraction of the information relating to the patient and/or the display of an image of the region of interest, etc.

1.2.2. Terminal

The terminal 2 allows the optional processing of certain medical data acquired by the probe 1 and/or the display of images of the region of interest.

The terminal 2 is for example a mobile terminal such as a mobile phone of Smartphone type, a personal assistant (or PDA, or Personal Digital Assistant) or any type of mobile terminal known to those skilled in the art, such as a connected watch of Apple Watch® type.

In a variant, the terminal 2 can be a virtual terminal, i.e. an emulation of a physical mobile terminal. In the context of this invention, the term “virtual terminal” encompasses any virtual resource, and/or a part of a real entity. For example, the virtual terminal can be a computer program, a virtual machine, an instance implemented in a “cloud computing” environment, a sub-system of a physical apparatus such as a display screen etc.

Besides the processing, where applicable, of data acquired by the probe 1 and/or display of the region of interest, the terminal 2 allows the transfer of:
data transmitted by the probe (medical data, monitoring data etc.) toward the platform 3 and the transfer of
data transmitted by the platform 3 (control data etc.) toward the probe 1.

The exchanges of data between the terminal 2 and the platform 3 are implemented using a computer network such as the Internet.

1.2.3. Remote Platform

The platform 3 makes it possible to:
Implementation of processes requiring computational power exceeding the capabilities of the terminal 2, and/or
storage of received data (medical data, monitoring data etc.), and/or
the driving of probe 1 by transmitting control instructions to it via the terminal 2.

The platform 3 further allows the generation of certificates for the probe and the terminal, as will be described in more detail in the remainder of the text.

The platform 3 comprises a processing unit, for example including one or more computers, one or more micro-computers, one or more workstations, and/or other devices known to those skilled in the art including one or more processors, one or more microcontrollers, one or more programmable automated systems, one or more application-specific integrated circuits, and/or other programmable circuits.

The platform 3 also comprises a storage unit including one (or more) memories which can be a ROM/RAM memory, a USB key, or a memory of a central server.

The processing unit can be integrated into or separate from the storage unit. Moreover, the different elements constituting the processing unit (or the storage unit respectively) can be located in physically different positions on the scale of a building, a city, a country or one or more continents.

Besides the retention of data associated with the medical examination (medical data, monitoring data etc.), the storage unit also makes it possible to store programming code instructions intended to execute certain steps of the authentication method described in the remainder of the text.

The same applies for the probe 1 and the terminal 2 which each comprise a respective memory for the storage of programming code instructions for the implementation of the authentication method explained below.

1.3. Trust Circles

The platform constitutes a certification authority making it possible to guarantee the origin of the certificate assigned to the probe on the one hand and to the terminal on the other.

More precisely, the platform 3 is characterized by:
a platform public key, known to the probe and to the terminal
a platform private key known only to the platform, and used to sign: the probe certificate assigned to the probe at the time of its manufacturing, and the terminal certificate assigned to the terminal at the time of the subscription by the user to a customer account.

The platform public key is recorded:
in a memory of the probe 1 at the time of its manufacturing, and
in a memory of the terminal 2 at the time of the subscription by the user to a customer account, for example following the transmission by the platform of a platform certificate containing: the platform public key, and a signature obtained on the basis of the platform private key.

Only the platform 3 has the platform private key making it possible to:
sign certificates addressed to the probe and to the terminal, and
decrypting the messages encrypted on the basis of the platform public key.

In other words, the platform private key is stored only in the storage unit of the platform 3.

Thus, the probe 1 and the terminal 2 can verify the authenticity of the certificates sent by platform 3 using the platform public key, and no software entity can substitute itself for platform 3 to generate fraudulent certificates.

1.3.1. First Trust Circle and Certificate

The probe 1 and the platform 3 are resources belonging to the same trust space. The probe 1 and the platform 3 are for example manufactured by a same organization, or belong to the same organization (same company or company group).

The storage unit of the platform 3 comprises a table classifying all the probes 1 manufactured by and/or belonging to the organization.

More precisely, each probe 1 is characterized by:
a probe identifier (a series of characters),
a known probe private key,
a probe public key used to encrypt messages for the attention of the probe 1, and
a probe certificate produced by the platform 3.

The probe certificate in particular comprises:
a probe identifier,
the probe public key, and
a signature obtained on the basis of the platform private key.

The probe identifier, the probe public and private keys, the probe certificate and the platform public key are stored in a memory of the probe at the time of its manufacturing.

The probe identifier, the probe public key and the probe certificate are stored in the probe table contained in the storage unit of the platform 3.

Only the probe 1 has the probe private key making it possible to decrypt the messages encrypted on the basis of the probe public key. In other words, the probe private key is stored only in the memory of the probe 1.

1.3.2. Second Trust Circle and Certificate

The terminal 2, meanwhile, does not belong to the same organization. It is able to operate with different probes 1. It belongs to a user having a customer account with the platform 3 and allowing the user to identify himself.

At the time of subscription to a customer account, a terminal identifier, a terminal private key, a terminal public key and a terminal certificate are generated. The terminal public and private keys are generated by the terminal, while the terminal certificate and identifier are generated by the platform 3.

More precisely, during the subscription to a customer account, the terminal 2 generates terminal public and private keys. The terminal public key is transmitted to the platform 3 in a subscription request message. In the scenario where the user of the terminal 2 has a probe 1 with which he wishes to perform an examination, the subscription request message can also comprise the identifier of the probe intended to be combined with the terminal to perform examinations. This allows the platform to associate the terminal with a probe of the probe table contained in the storage unit. As will be described in more detail in the remainder of the text, such an association makes it possible to dispense with the need for the probe or the terminal to transmit to the platform a session key generated after the identification protocol described below. In the scenario where a user of the terminal 2 does not have a probe 1 at the time of the subscription, or if he has several probes, the identifier of the probe intended to be combined with the terminal to perform an examination can be sent to the platform after the subscription to a customer account, particularly a few minutes before the implementation of an examination.

In response to the subscription request message, the platform 3 generates a terminal identifier, and produces a terminal certificate including:
the terminal identifier,
the terminal public key, and
a signature obtained on the basis of the platform private key.

This certificate is sent to the terminal. It can be encrypted on the basis of the terminal public key. The platform certificate including the platform public key is also sent to the terminal.

The terminal identifier, the terminal public and private keys, the platform public key and the terminal certificate are stored in the memory of the terminal 2. The terminal identifier, the terminal public key and the terminal certificate are retained in a table stored in the storage unit of the platform 3. In the scenario where the probe identifier is also transmitted to the platform, this platform stores in a probe/terminal correspondence table the identifiers of the probe and of the terminal that must be combined for the implementation of an examination session.

2. Authentication Method

A more detailed description will now follow of the operating principle of the authentication method according to the invention.

It will be supposed in the remainder of the text that the user has previously subscribed to a user account with the platform 3, and that the terminal 2 of the user has been recorded at the time of the subscription to the user account. During the recording operation, terminal public and private keys have been generated by the terminal 2;
the terminal private key allows the terminal 2 to decrypt the received messages that have been encrypted using the terminal public key,
the terminal public key allows the entities holding the terminal certificate to encrypt the messages addressed to the terminal.

Also at the time of the recording operation, the platform certificate and a terminal certificate have been sent to the terminal 2 by the remote platform, and stored in the memory of the terminal. The platform certificate particularly contains the platform public key; this platform public key allows the terminal to verify the authenticity of the certificates produced by the platform, and where applicable to encrypt the messages for the platform 3. The terminal certificate contains:
the identifier of the terminal,
the terminal public key which allows the entities holding the terminal certificate to encrypt the messages addressed to the terminal,
a signature obtained on the basis of the platform private key; this signature makes it possible to authenticate the platform as the certificate entity that generated the certificate.

As indicated above:
the memory of the probe 1 contains:
   the probe private key making it possible to decrypt messages encrypted with the probe public key,
   the platform public key, where applicable making it possible to encrypt messages addressed to the platform 3 and to verify the authenticity of the certificates transmitted by the platform, and
   the probe certificate containing:
      the probe identifier,
      the probe public key,
      a signature obtained on the basis of the platform private key;
the memory of the terminal 2 contains:
   the terminal private key used to decrypt messages encrypted with the terminal public key,
   the certificate of the platform containing the platform public key, which where applicable makes it possible to encrypt the messages addressed to the platform 3 and to verify the authenticity of the signature of the certificates transmitted by the platform, and
   the terminal certificate containing:
      the terminal identifier,
      the terminal public key,
      a signature obtained on the basis of the platform private key;
the storage unit of the terminal 3 contains:
   a probe table including the identifier of each probe 1 of the organization and the probe certificate associated with each probe identifier,
   a terminal including the identifier of each terminal 2 and the terminal certificate associated with each terminal identifier,
   a probe/terminal correspondence table including the probe and terminal identifiers that must be combined for the implementation of an examination session,
   the platform private key used to sign the certificates and decrypt the messages encrypted with the platform public key.

The authentication method comprises two phases:
a first phase of dialog 100 between the terminal 2 and the probe 1 to allow the probe to authenticate the terminal, and
a second phase of dialog 300 between the terminal 2 and the probe 1 to allow the terminal to authenticate the probe.

2.1. Before the Implementation of the Authentication Method

When the user wishes to perform an examination, he enters on the entry means of the terminal 2 information concerning the examination, and in particular the identifier of the probe intended to be used for the examination.

This information and other information such as:
the identifier of the terminal,
personal data relating to the patient (first name, surname, case number etc.)
data related to the examination (acquisition date of the examination data, type of examination etc.)

are incorporated into an examination request message.

This examination request message is sent to the platform 3 which records it in the storage unit and updates the probe/terminal correspondence table by associating with it the probe and terminal identifiers.

Advantageously, the examination request message can be encrypted on the basis of the platform public key. This makes it possible to limit the risks of obtainment of critical information by a malicious third party who has intercepted all the communications, for example to usurp the identity of the terminal 2.

The platform 3 verifies that the user has system user rights according to the terminal identifier. If the user has user rights, the platform emits a pairing authorization message, otherwise the platform emits an error message prohibiting the pairing.

Advantageously, the authorization message transmitted by the platform 3 can be encrypted using the terminal public key. The fact of encrypting the authorization message using the terminal public key makes it possible to avoid the risk of fraudulent interception of information critical to the system, this information being encrypted and therefore unusable in that state. This moreover allows the platform to ensure that the terminal 2 that generated the request and the terminal associated with the identifier contained in the request do indeed constitute one and the same entity (only the terminal, the identifier of which has been indicated in the request, having the terminal private key making it possible to decrypt the message of the platform).

When the terminal has received the authorization message, the probe 1 and the terminal 2 implement the first dialog phase 100.

2.2. First Dialog Phase

As indicated previously, the first dialog phase 100 allows the probe 1 to authenticate the terminal 2.

In a first step, the terminal 2 emits 110 a pairing request addressed to the probe 1. This pairing request contains the terminal certificate which will be used by the probe 1 to verify that the terminal is indeed a trusted entity.

The probe 1 receives 120 the pairing request, and extracts from it the terminal certificate.

The probe 1 verifies 130 the authenticity of the terminal certificate by comparing the signature of the terminal certificate with the platform public key stored in the memory at the time of its manufacturing.

If the terminal certificate is authentic, the probe 1 extracts 140 the terminal public key contained in the terminal certificate and records it in its internal memory. This terminal key will be used to generate a “session key” as will be described in more detail in the remainder of the text. If the terminal certificate is not authentic, an error message is transmitted 135.

The probe 1 generates verification information (for example a series of random figures), encrypts them using the terminal public key, and incorporates them into an answer message. This answer message is sent 150 by the probe 1 to the terminal 2.

The terminal 2 receives 160 the answer message and decrypts the verification information using the terminal private key. This terminal private key, known solely to the terminal 2, is the only one that can decrypt the verification information. Specifically, as recalled in point 1.1.1, in the case of an asymmetrical encryption, information encrypted using a public key cannot be decrypted using this same public key: only the private key associated with this public key makes it possible to decrypt this information.

The terminal 2 incorporates the verification information into a confirmation message. The confirmation message is sent 170 by the terminal 2 to the probe 1.

The probe 1 receives 180 the confirmation message and extracts the verification information from the confirmation message.

The probe 190 then compares:
the verification information of the confirmation message,
the verification information contained in the answer message transmitted by the probe 1.

If the comparison is positive (the verification information of the confirmation and answer messages match), the probe 1 emits 200 an authentication validation message addressed to the terminal 2. The second dialog phase 300 can be implemented.

If the comparison is negative (the verification information of the confirmation and answer messages do not match), the probe 1 emits 195 an error message and refuses the pairing between the probe 1 and the terminal 2.

The first dialog phase 100 therefore allows the probe to authenticate the terminal 2 using the terminal certificate including the terminal public key:
the verification of the signature of this certificate—using the platform public key—allows the probe to confirm that the certificate has indeed been transmitted by a trusted authority (i.e. the platform),
the exchange of verification information encrypted on the basis of the terminal public key makes it possible to confirm that the entity that addressed this certificate to it is indeed the entity for which this certificate was produced by the trusted authority.

2.3. Second Dialog Phase

As indicated previously, the second dialog phase 300 allows the terminal 2 to authenticate the probe 1.

In a first step, the terminal 2 emits 310 a certificate request message addressed to the probe 1.

The probe 1 receives 320 the certificate request message and generates a result message into which the probe certificate is incorporated. Advantageously, the result message can be encrypted using the terminal public key in order to limit the risks of interception of the information it contains by a fraudulent trusted third-party entity.

The probe 1 sends 330 the result message addressed to the terminal 2

The terminal 2 receives the result message, decrypts it and extracts the probe certificate. The terminal 2 verifies 340 the authenticity of the probe certificate by comparing the signature of the probe certificate with the platform public key stored in the memory at the time of the subscription to the customer account.

If the probe certificate is authentic, the terminal 1 extracts 350 the probe public key contained in the probe certificate and records it in its internal memory. This probe key will be used to generate the “session key”. If the terminal certificate is not authentic, an error message is sent 345.

The terminal 2 generates verification information (for example a series of random figures), encrypts the verification information using the probe public key, and incorporates them into a justification message. This justification message is sent 360 by the terminal 2 to the probe 1.

The probe 1 receives 370 the justification message and decrypts the verification information using the probe private key known only to the probe 1.

The probe 1 incorporates the verification information into a proof message. The proof message is sent 380 by the probe to the terminal 2.

The terminal receives 390 the proof message and extracts the verification information from the proof message.

The terminal then compares 400:
the verification information of the proof message,
with the verification information contained in the justification message previously transmitted by the terminal 2.

If the comparison is positive (the verification information of the messages match), the terminal 2 emits 410 an authentication validation message for the probe 1. The probe and the terminal are paired.

If the comparison is negative (the verification information of the messages do not match), the terminal 2 emits 405 an error message and refuses the pairing between the probe 1 and the terminal 2.

3. Examination

Once the first and second dialog phases 100, 300 have been carried out and the successful authentication made, the probe 1 and the terminal 2 are paired. A pairing confirmation message can be sent by the probe 1 or the terminal 2 to the platform 3.

Each entity of the system then generates the session key on the basis of the probe and terminal public keys. Specifically, the probe and terminal public keys are stored:
in the memory of the probe (the probe public key is recorded at the time of manufacturing of the probe, and the terminal public key is recorded at the time of implementation of the first dialog phase 100),
in the memory of the terminal (the probe public key is recorded in the memory of the terminal 2 at the time of implementation of the second dialog phase, and the terminal public key is recorded in the memory of the terminal at the time of subscription to the customer account)
in the storage unit of the platform (the probe and terminal public keys are contained in the probe and terminal tables, and the probe/terminal correspondence table is used to define which probe is associated with each terminal).

Thus, one and the same session key is generated independently by the probe, the terminal and the platform. This session key is therefore not transmitted between the different entities, which limits subsequent fraud risks.

The session key is used to encrypt/decrypt the data exchanged according to a symmetrical cryptography mode (the session key is used both to encrypt and decrypt the data). The session key will be used during the implementation of the examination to:
encrypt/decrypt the messages addressed to/received from the probe 1
encrypt/decrypt the messages addressed to/received from the terminal 2,
encrypt/decrypt the messages addressed to/received from the platform 3.

The duration of validity of the session key depends on the type of application concerned. It can be of a few tens of minutes for an examination for a patient, or of several hours/days for an imaging session in an emergency vehicle (in mobility).

The session key guarantees:
the integrity of the system data on the one hand, and the confidentiality of the medical data on the other: only the authenticated probe 1, terminal 2 and platform 3 have the session key allowing access to the data, and
the reliability of the medical data: the session key is unique for each examination (thus if several successive examinations are performed by one and the same user for one and the same patient, there is no risk of confusion in the data associated with these different examinations, the correspondence between the session key and the examination being biunivocal).

Advantageously, the probe public and private keys may be used for the exchange of items of sensitive information between the platform 3 and the probe 1, via the terminal 2, without the terminal having access to these items of sensitive information. These items of sensitive information for example consist in instructions to drive the probe. Specifically, the sequence (or sequences) of configuration of the probe (for the acquisition of the data as part of the examination) cannot be sent directly from the platform 3 to the probe 1, particularly due to the limited memory capacity of the probe 1. This is why the terminal 2 can be used to store this (or these) sequence (or sequences), and to transmit it (or them) sequentially in pieces to the probe 1. By asymmetrically encrypting these driving instructions using the probe public and private keys, it is possible to transmit them via the terminal without it being able to access them. The fact that the terminal cannot access the driving instructions encrypted asymmetrically (on the basis of the probe public and private keys) makes it possible to guarantee the integrity of the data driving the deposition of ultrasonic energy in the biological tissue of the patient.

The end of the examination can be scheduled by the user by using the terminal 2. In this case, the terminal 2 sends to the probe 1 and to the platform 3 an end-of-examination command message. If certain medical data relating to the examination have not been acquired by the probe 1 and/or have not been processed by the platform 3, the probe 1 and the platform 3 can send an acceptance message indicating that the end-of-examination command has indeed been taken into account and that this will be effective from the moment of completion of the acquisition and/or processing of the medical data by the probe 1 and/or the platform 3.

4. Conclusions

The previously-defined invention allows the secure and reliable exchange of data between different authenticated entities of a system using an Internet-type network.

It will be understood that many modifications may be made to the previously-described invention without materially departing from the new teachings and advantages described here.

Consequently, all modifications of this type are intended to be made within the scope of the attached claims.

1. A management method for managing data exchanges during a procedure of medical examination of a patient, the management method allowing the management of the data exchanges between:
a data acquisition probe, wherein said probe includes a memory containing a probe digital certificate including a probe public key,
a terminal configured to communicate with the probe via wired or wireless communication means, wherein said terminal includes a memory containing a terminal digital certificate including a terminal public key,
a remote platform configured to communicate with the terminal via a computer network, wherein said platform is configured to:
issue the probe digital certificate to the probe,
issue the terminal digital certificate to the terminal,
wherein the method comprises, prior to the implementation of the examination procedure, the implementation of an authentication procedure comprising the following phases:
a first phase wherein the probe verifies the identity of the terminal on the basis of the terminal digital certificate, and
a second phase wherein the terminal verifies the identity of the probe on the basis of the probe digital certificate,
a third phase wherein the probe, the terminal and the platform each generate an identical session key, wherein said session key is produced on the basis of the probe and terminal public keys.

2. The management method as claimed in claim 1, wherein the identical session key is generated independently by the probe, the terminal and the platform, wherein the probe and terminal public keys are stored respectively:
in the memory of the probe,
in the memory of the terminal, and
in a storage unit of the platform, and wherein said session key is not exchanged between the probe, the terminal and the platform via the communication means and/or the computer network.

3. The management method as claimed in claim 1, wherein the session key is used to encrypt according to a symmetrical cryptography mode data exchanged between the probe, the terminal and the platform during the implementation of the examination.

4. The management method as claimed in claim 1, wherein the first phase comprises the following steps:
transmission by the terminal of a pairing request, wherein the pairing request contains the terminal digital certificate,
reception by the probe of the pairing request and extraction of the terminal digital certificate, and
verification by the probe of the terminal digital certificate by certifying the authenticity of said terminal digital certificate using a platform public key contained in the memory of the probe.

5. The management method as claimed in claim 4, wherein the first phase further comprises the following steps:
if the terminal digital certificate is authentic, then exchange of verification information between the probe and the terminal, wherein said verification information being is successively encrypted and decrypted using the terminal public key and a terminal private key stored in the memory of the terminal,
if the terminal digital certificate is not authentic, then transmission, by the probe, of an alert message.

6. The management method as claimed in claim 5, wherein the step of exchanging verification information comprises the sub-steps consisting in:
extraction, by the probe, of the terminal public key contained in the terminal digital certificate,
generation, by the probe, of a verification information,
asymmetrical encryption, by the probe, of the verification information with the terminal public key,
sending, by the probe, of an answer message, wherein said answer message includes the verification information encrypted with the terminal public key,
reception, by the terminal, of the answer message and decryption of the verification information using a terminal private key stored in the memory of the terminal,
sending, by the terminal, of a confirmation message, wherein said confirmation message contains the decrypted verification information,
reception, by the probe, of the confirmation message,
comparison, by the probe, of the decrypted verification information contained in the confirmation message with the verification information contained in the answer message transmitted by the probe, to define whether said verification information are identical or different, and
if the verification information are identical, transmission of a validation message representative of a successful authentication,
if the verification information are different, transmission of an alert message representative of a failed authentication.

7. The management method as claimed in claim 1, wherein the second phase further comprises the following steps:
reception, by the terminal, of a result message transmitted by the probe, wherein the probe digital certificate is incorporated into the result message,
extraction, by the terminal, of the probe digital certificate,
verification by the terminal of the authenticity of the probe digital certificate by comparing the signature of said terminal digital certificate with a platform public key contained in the memory of the terminal.

8. The management method as claimed in claim 7, wherein the second phase further comprises the following steps:
if the probe digital certificate is authentic, then exchange of verification information between the probe and the terminal, wherein said verification information is successively encrypted and decrypted using the probe public key and a probe private key stored in a memory of the probe,
if the probe digital certificate is not authentic, then transmission, by the terminal, of an alert message.

9. The management method as claimed in claim 8, wherein the step of exchange of verification information comprises the sub-steps consisting in:
extraction, by the terminal, of the probe public key contained in the probe digital certificate,
generation, by the terminal, of a verification information,
asymmetrical encryption, by the terminal, of the verification information using the probe public key,
sending, by the terminal, of a justification message, wherein said justification message includes the verification information encrypted with the probe public key,
reception, by the probe, of the justification message and decryption of the verification information using a probe private key stored in the memory of the probe,
sending, by the probe, of a proof message containing the decrypted verification information,
reception, by the terminal, of the proof message,
comparison, by the terminal, of the verification information contained in the proof message with the verification information contained in the justification message transmitted by the terminal, to define whether said verification information are identical or different, and
if the verification information are identical, transmission of a validation message representative of a successful authentication,
if the verification information are different, transmission of an alert message representative of a failed authentication.

10. The management method as claimed in claim 1, wherein the method comprises, prior to the implementation of an authentication procedure, a subscription procedure wherein:
the terminal sends the platform an examination request message including a probe identifier, a terminal identifier, and the terminal public key,
the platform sends the terminal a pairing authorization message including a platform certificate which comprises including a platform public key and a terminal certificate which comprises the terminal public key.

11. A system for the exchange of data during a procedure of examination of a patient, the system comprising:
a data acquisition probe, said probe including a memory containing a probe digital certificate including a probe public key,
a terminal configured to communicate with the probe via wired or wireless communication means, wherein said terminal includes a memory containing a terminal digital certificate including a terminal public key,
a remote platform configured to communicate with the terminal via a computer network, wherein said platform is configured to:
issue the probe digital certificate to the probe,
issue the terminal digital certificate to the terminal,
wherein the probe comprises a calculator, and the terminal and the platform comprise processors configured to implement an authentication procedure, prior to the implementation of the examination procedure, the authentication procedure comprising the following phases:
a first phase wherein the probe verifies the identity of the terminal on the basis of the terminal digital certificate, and
a second phase wherein the terminal verifies the identity of the probe on the basis of the probe digital certificate,
a third phase wherein the probe, the terminal and the platform each generate an identical session key, said session key being produced on the basis of the probe and terminal public keys.

12. (canceled)

13. The system as claimed in claim 11, wherein the identical session key is generated independently by the probe's calculator, the terminal's processor and the platform's processor, wherein the probe and terminal public keys are stored respectively:
in the memory of the probe,
in the memory of the terminal, and
in a storage unit of the platform,
and wherein said session key is not exchanged between the probe, the terminal and the platform via the communication means and/or the computer network.

14. The system as claimed in claim 11, wherein the session key is used to encrypt according to a symmetrical cryptography mode data exchanged between the probe, the terminal and the platform during the implementation of the examination.

15. The system as claimed in claim 11, wherein the first phase comprises the following steps:
transmission by the terminal of a pairing request, wherein the pairing request contains the terminal digital certificate,
reception by the probe of the pairing request and extraction of the terminal digital certificate,
verification by the probe of the terminal digital certificate by certifying the authenticity of said terminal digital certificate using a platform public key contained in the memory of the probe.

16. The system as claimed in claim 15, wherein the first phase further comprises the following steps:
if the terminal digital certificate is authentic, then exchange of verification information between the probe and the terminal, wherein said verification information is successively encrypted and decrypted using the terminal public key and a terminal private key stored in the memory of the terminal,
if the terminal digital certificate is not authentic, then transmission, by the probe, of an alert message.

17. The system as claimed in claim 16, wherein the step of exchanging verification information comprises the sub-steps consisting in:
extraction, by the probe, of the terminal public key contained in the terminal digital certificate,
generation, by the probe, of a verification information,
asymmetrical encryption, by the probe, of the verification information with the terminal public key,
sending, by the probe, of an answer message, wherein said answer message includes the verification information encrypted with the terminal public key,
reception, by the terminal, of the answer message and decryption of the verification information using a terminal private key stored in the memory of the terminal,
sending, by the terminal, of a confirmation message, wherein said confirmation message contains the decrypted verification information,
reception, by the probe, of the confirmation message,
comparison, by the probe, of the decrypted verification information contained in the confirmation message with the verification information contained in the answer message transmitted by the probe, to define whether said verification information are identical or different, and
if the verification information are identical, transmission of a validation message representative of a successful authentication,
if the verification information are different, transmission of an alert message representative of a failed authentication.

18. The system as claimed in claim 11, wherein the second phase further comprises the following steps:
reception, by the terminal, of a result message transmitted by the probe, wherein the probe digital certificate is incorporated into the result message,
extraction, by the terminal, of the probe digital certificate,
verification by the terminal of the authenticity of the probe digital certificate by comparing the signature of said terminal digital certificate with a platform public key contained in the memory of the terminal.

19. The system as claimed in claim 18, wherein the second phase further comprises the following steps:
if the probe digital certificate is authentic, then exchange of verification information between the probe and the terminal, wherein said verification information is successively encrypted and decrypted using the probe public key and a probe private key stored in a memory of the probe,
if the probe digital certificate is not authentic, then transmission, by the terminal, of an alert message.

20. The system as claimed in claim 19, wherein the step of exchange of verification information comprises the sub-steps consisting in:
extraction, by the terminal, of the probe public key contained in the probe digital certificate,
generation, by the terminal, of a verification information,
asymmetrical encryption, by the terminal, of the verification information using the probe public key,
sending, by the terminal, of a justification message, wherein said justification message includes the verification information encrypted with the probe public key,
reception, by the probe, of the justification message and decryption of the verification information using a probe private key stored in the memory of the probe,
sending, by the probe, of a proof message containing the decrypted verification information,
reception, by the terminal, of the proof message,
comparison, by the terminal, of the verification information contained in the proof message with the verification information contained in the justification message transmitted by the terminal, to define whether said verification information are identical or different, and
if the verification information are identical, transmission of a validation message representative of a successful authentication,
if the verification information are different, transmission of an alert message representative of a failed authentication.
